SEQUENCE LISTING (34 sequences)

SEQ ID NO: 1 - RNA, 31 nt, synthetic construct
  source 1..31: mol_type = other RNA
gtcgttgtagtatagtggtaagtattcccgc

SEQ ID NO: 2 - RNA, 31 nt, synthetic construct
  source 1..31: mol_type = other RNA
tccgttgtagtctagctggtcaggatactcg

SEQ ID NO: 3 - RNA, 31 nt, synthetic construct
  source 1..31: mol_type = other RNA
tccgatgtcgtccagcggttaggatatctgg

SEQ ID NO: 4 - RNA, 31 nt, synthetic construct
  source 1..31: mol_type = other RNA
gcgtctgtagtccaacggttaggataattgc

SEQ ID NO: 5 - RNA, 31 nt, synthetic construct
  source 1..31: mol_type = other RNA
gcgggaatacttaccactatactacaacgac

SEQ ID NO: 6 - DNA, 31 bp, synthetic construct
  source 1..31: mol_type = other DNA
gcgggaatacttaccactatactacaacgac

SEQ ID NO: 7 - DNA, 13 bp, synthetic construct
  source 1..13: mol_type = other DNA
gtcgttgtagtat

SEQ ID NO: 8 - RNA, 13 nt, synthetic construct
  source 1..13: mol_type = other RNA
gtcgttgtagtat

SEQ ID NO: 9 - RNA, 31 nt, synthetic construct
  source 1..31: mol_type = other RNA
cgagtatcctgaccagctagactacaacgga

SEQ ID NO: 10 - DNA, 31 bp, synthetic construct
  source 1..31: mol_type = other DNA
cgagtatcctgaccagctagactacaacgga

SEQ ID NO: 11 - RNA, 16 nt, synthetic construct
  source 1..16: mol_type = other RNA
gctagactacaacgga

SEQ ID NO: 12 - DNA, 16 bp, synthetic construct
  source 1..16: mol_type = other DNA
gctagactacaacgga

SEQ ID NO: 13 - RNA, 31 nt, synthetic construct
  source 1..31: mol_type = other RNA
ccagatatcctaaccgctggacgacatcgga

SEQ ID NO: 14 - DNA, 31 bp, synthetic construct
  source 1..31: mol_type = other DNA
ccagatatcctaaccgctggacgacatcgga

SEQ ID NO: 15 - RNA, 18 nt, synthetic construct
  source 1..18: mol_type = other RNA
ccgctggacgacatcgga

SEQ ID NO: 16 - DNA, 18 bp, synthetic construct
  source 1..18: mol_type = other DNA
ccgctggacgacatcgga

SEQ ID NO: 17 - RNA, 31 nt, synthetic construct
  source 1..31: mol_type = other RNA
gcaattatcctaaccgttggactacagacgc

SEQ ID NO: 18 - DNA, 31 bp, synthetic construct
  source 1..31: mol_type = other DNA
gcaattatcctaaccgttggactacagacgc

SEQ ID NO: 19 - RNA, 15 nt, synthetic construct
  source 1..15: mol_type = other RNA
ttggactacagacgc

SEQ ID NO: 20 - DNA, 15 bp, synthetic construct
  source 1..15: mol_type = other DNA
ttggactacagacgc

SEQ ID NO: 21 - RNA, 14 nt, synthetic construct
  source 1..14: mol_type = other RNA
gtcgttgtagtata

SEQ ID NO: 22 - RNA, 17 nt, synthetic construct
  source 1..17: mol_type = other RNA
gtggtaagtattcccgc

SEQ ID NO: 23 - RNA, 16 nt, synthetic construct
  source 1..16: mol_type = other RNA
tccgttgtagtctagc

SEQ ID NO: 24 - RNA, 15 nt, synthetic construct
  source 1..15: mol_type = other RNA
tggtcaggatactcg

SEQ ID NO: 25 - RNA, 18 nt, synthetic construct
  source 1..18: mol_type = other RNA
tccgatgtcgtccagcgg

SEQ ID NO: 26 - RNA, 13 nt, synthetic construct
  source 1..13: mol_type = other RNA
ttaggatatctgg

SEQ ID NO: 27 - RNA, 15 nt, synthetic construct
  source 1..15: mol_type = other RNA
gcgtctgtagtccaa

SEQ ID NO: 28 - RNA, 15 nt, synthetic construct
  source 1..15: mol_type = other RNA
cggttaggataattg

SEQ ID NO: 29 - RNA, 71 nt, unidentified
  source 1..71: mol_type = tRNA
gtcgttgtagtatagtggtaagtattcccgcctgtcacgcgggtgaccgggttcgatccccggcaacggcg

SEQ ID NO: 30 - DNA, 40 bp, unidentified
  source 1..40: mol_type = genomic DNA
attgacaacctaaaatactacaacgatatgattggtgttt

SEQ ID NO: 31 - DNA, 40 bp, unidentified
  source 1..40: mol_type = genomic DNA
attgacaaccttcagtactacaacgatatgatagtgcttt

SEQ ID NO: 32 - DNA, 40 bp, unidentified
  source 1..40: mol_type = genomic DNA
attgacaacctaaaatactacaacgacatgattggtgttt

SEQ ID NO: 33 - DNA, 40 bp, unidentified; identical to SEQ ID NO: 32
  source 1..40: mol_type = genomic DNA

SEQ ID NO: 34 - DNA, 40 bp, unidentified
  source 1..40: mol_type = genomic DNA
attgacaaccttcagtactacaacgacatgatagtgtttt

(19) United States
(12) Patent Application Publication
(10) Pub. No.: US 2023/0112054 A1
(43) Pub. Date: Apr. 13, 2023

(54) MODULATING STRESS RESPONSES BY A NOVEL CHROMATIN-ASSOCIATED GUIDE RNA DERIVED FROM TRANSFER RNA

(71) Applicant: TEXAS STATE UNIVERSITY, San Marcos, TX (US)

(72) Inventors: Hong-Gu Kang, Buda, TX (US); Sung-II Kim, Bryan, TX (US)

(73) Assignee: TEXAS STATE UNIVERSITY, San Marcos, TX (US)

(21) Appl. No.: 17/965,436

(22) Filed: Oct. 13, 2022

Related U.S. Application Data
(60) Provisional application No. 63/255,334, filed on Oct. 13, 2021.
(60) Provisional application No. 63/274,807, filed on Nov. 2, 2021.

(51) Int. Cl. C12N 15/11; C12N 15/113

(52) U.S. Cl. CPC C12N 15/111; C12N 15/113

(57) ABSTRACT

Embodiments of the present disclosure pertain to methods of modulating a stress response in a eukaryotic organism against an environmental stress by introducing an RNA fragment (RF) or an antisense nucleotide to the eukaryotic organism. Thereafter, the RF or antisense nucleotide modulates the stress response against the environmental stress. Additional embodiments of the present disclosure pertain to the RFs and antisense nucleotides of the present disclosure. The RFs generally include: a sequence recognition site that contains a reverse complement sequence of a nucleotide sequence of the organism; and a stem loop structure with a paired region that includes paired RNA nucleotides, and an unpaired region that includes unpaired RNA nucleotides in the form of a loop. The antisense nucleotides may include a reverse complement sequence of the RFs.

### CROSS-REFERENCE TO RELATED APPLICATIONS

The present application claims the benefit of U.S. Provisional Patent Application No. 63/255,334, filed on Oct. 13, 2021, and U.S. Provisional Patent Application No. 63/274,807, filed on Nov. 2, 2021. The entirety of each of the aforementioned applications is incorporated herein by reference.

### STATEMENT REGARDING FEDERALLY SPONSORED RESEARCH

This invention was made with government support under 1553613 awarded by the National Science Foundation. The government has certain rights in the invention.

Pursuant to 37 C.F.R. § 1.834, Applicant has submitted a sequence listing as an XML file (“Sequence Listing”). The name of the file containing the Sequence Listing is “VR39977.P072US.xml”. The date of the creation of the Sequence Listing is Oct. 13, 2022. The size of the Sequence Listing is 30,000 bytes. Applicant hereby incorporates by reference the material in the Sequence Listing.

### BACKGROUND

Current methods and compounds for treating or preventing environmental stresses (e.g., infections) of eukaryotic organisms (e.g., plants) have numerous limitations. Various embodiments of the present disclosure aim to address such limitations.

### SUMMARY

In some embodiments, the present disclosure pertains to methods of modulating a stress response in a eukaryotic organism against an environmental stress by introducing an RNA fragment (RF) or an antisense nucleotide to the eukaryotic organism. Thereafter, the RF or antisense nucleotide modulates the stress response against the environmental stress.

In some embodiments, the RF includes a sequence recognition site that contains a reverse complement sequence of a nucleotide sequence of the organism. In some embodiments, the RF also includes a stem loop structure with a paired region that includes paired RNA nucleotides, and an unpaired region that includes unpaired RNA nucleotides in the form of a loop.

In some embodiments, the RF includes, without limitation, 5′ GUCGUUGUAGUAUAGUGGUAAGUAUUCCCGC-3′ (SEQ ID NO: 1), 5′-UCCGUUGUAGUCUAGCUGGUCAGGAUACUCG-3′ (SEQ ID NO: 2), 5′-UCCGAUGUCGUCCAGCGGUUAGGAUAUCUGG-3′ (SEQ ID NO: 3), 5′-GCGUCUGUAGUCCAACGGUUAGGAUAAUUGC-3′ (SEQ ID NO: 4), or a sequence with at least 75% identity with any one of SEQ ID NOS: 1-4.

In some embodiments, the RF includes 5′ GUCGUUGUAGUAUAGUGGUAAGUAUUCCCGC-3′ (SEQ ID NO: 1). In some embodiments, 5′-GUCGUUGUAGUAUA-3′ represents the sequence recognition site, 5′-GUGGUAAGUAUUCCCGC-3′ represents the stem loop structure, 5′-GUGG-3′ pairs with 5′-CCGC-3′ to form the paired region of the stem loop structure, and 5′-UAAGUAUUC-3′ represents the loop of the stem loop structure.

In some embodiments, the antisense nucleotide includes a sequence that is complementary to the reverse complement sequence of the RF. In some embodiments, the antisense nucleotide is an RNA or DNA nucleotide. In some embodiments, the antisense nucleotide includes, without limitation, a reverse complement sequence of SEQ ID NO: 1, 5′-GCGGGAAUACUUACCACUAUACUACAACGAC-3′ (SEQ ID NO: 5), 5′-GCGGGAATACTTACCACTATACTACAACGAC-3′ (SEQ ID NO: 6), an antisense nucleotide for a sequence recognition site of SEQ ID NO: 1, 5′-GTCGTTGTAGTAT-3′ (SEQ ID NO: 7), 5′-GUCGUUGUAGUAU-3′ (SEQ ID NO: 8), a reverse complement sequence of SEQ ID NO: 2, 5′-CGAGUAUCCUGACCAGCUAGACUACAACGGA-3′ (SEQ ID NO: 9), 5′-CGAGTATCCTGACCAGCTAGACTACAACGGA-3′ (SEQ ID NO: 10), an antisense nucleotide for a sequence recognition site of SEQ ID NO: 2, 5′-GCUAGACUACAACGGA-3′ (SEQ ID NO: 11), 5′-GCTAGACTACAACGGA-3′ (SEQ ID NO: 12), a reverse complement sequence of SEQ ID NO: 3, 5′-CCAGAUAUCCUAACCGCUGGACGACAUCGGA-3′ (SEQ ID NO: 13), 5′-CCAGATATCCTAACCGCTGGACGACATCGGA-3′ (SEQ ID NO: 14), an antisense nucleotide for a sequence recognition site of SEQ ID NO: 3, 5′-CCGCUGGACGACAUCGGA-3′ (SEQ ID NO: 15), 5′-CCGCTGGACGACATCGGA-3′ (SEQ ID NO: 16), a reverse complement sequence of SEQ ID NO: 4, 5′-GCAAUUAUCCUAACCGUUGGACUACAGACGC-3′ (SEQ ID NO: 17), 5′-GCAATTATCCTAACCGTTGGACTACAGACGC-3′ (SEQ ID NO: 18), an antisense nucleotide for a sequence recognition site of SEQ ID NO: 4, 5′-UUGGACUACAGACGC-3′ (SEQ ID NO: 19), or 5′-TTGGACTACAGACGC-3′ (SEQ ID NO: 20).

In some embodiments, the eukaryotic organism is a plant or seed, such as a dicot plant or seed. In some embodiments, the stress response includes an immune response against the environmental stress. In some embodiments, the environmental stress includes pathogenesis, such as pathogenesis caused by a bacterial pathogen. In some embodiments, the method is utilized to treat or prevent the effect of the environmental stress (e.g., an infection) in the eukaryotic organism.

Additional embodiments of the present disclosure pertain to the RFs of the present disclosure. In some embodiments, the RFs of the present disclosure include at least one of SEQ ID NOS: 1-4. Further embodiments of the present disclosure pertain to the antisense nucleotides of the present disclosure.

### FIGURES

FIG. 1 illustrates a method of modulating a stress response in a eukaryotic organism.

FIGS. 2A-2B depict structures of a transfer RNA (tRNA) (FIG. 2A) and an RNA fragment derived from the tRNA (tRF) (FIG. 2B).

FIGS. 3A-3E illustrate that effector-triggered immunity (ETI) to Pseudomonas syringae pv. tomato (Pst) in Arabidopsis is associated with DCL1 and increased nuclear-localized tRNAs. FIG. 3A shows bacterial growth in wild type (WT) and dcl1-7 at 0 and 3 day-post-infection (dpi) with Pst containing or lacking AvrRpt2 (avrPst or virPst, respectively); rps2 is an ETI control. The mean±RSE (n≥9) from three independent experiments are presented. FIG. 3B shows relative PRI transcription levels measured by qRT-PCR, which are presented as mean±SE (n=3). FIG. 3C shows size distributions of nuclear sRNAs (11-100 nts) in WT and dcl1-7 at 1 hour-postinfection (hpi) with avrPst or buffer (mock). The y-axis represents a normalized read count in RPM. FIG. 3D shows percentage of tRNA-derived reads in 71-100 nt sRNAs. FIG. 3E shows normalized read counts of tRNA-derived 71-100 nt RNAs. Significance between indicated pairs is noted. *P<0.05; **P<0.01; ***P<0.001 (t-test).

FIGS. 4A-4F show that AGO2-associated 5′-tRNA fragments (tRFs) display DCL1dependent induction in Arabidopsis. FIGS. 4A-4C show sRNA-seq analysis of nuclear 5′-tRFs in avrPst- or mock-inoculated WT and dcl1-7 at 1 hpi. FIGS. 4D-4F show nuclear AGO-associated 5′-tRFs in avrPst-, virPst-, or mock-inoculated HA-AGO1 and HA-AGO2 transgenic Arabidopsis at 1 hpi, which were identified by sRIP (RNA immunoprecipitation)-seq via α-HA. WT was the negative control. The y-axis represents a normalized read count in RPM. FIGS. 4A and 4D show size distribution of 5′-tRFs. Normalized read counts of 31 nt-long 5′-tRFs originated from each tRNA isodecoder (FIGS. 4B and 4E) and of a representative avrPst-inducible, DCL1-dependent 5′-tRF (tRF^{31Asp2}) (FIGS. 4C and 4F). The mean±SE is presented (n=3, FIGS. 4A-4C; n=2, FIGS. 4D-4F). Significance from mock noted for highlighted samples only in broken line square border; *P<0.05; **P<0.01; ***P<0.001 (t-test).

FIGS. 5A-5G provide data illustrating that tRF^{31Asp2}induces immunity in an AGO2-clade dependent manner. FIG. 5A shows PR1 expression in local and distal leaves at the indicated times after tRF^{31Asp2} (1 μM) or buffer infiltration was examined by qRT-PCR. FIG. 5B shows bacterial growth in WT, dcl1-7, and ago2/3/7 pretreated with tRF^{31Asp2}, tRF^{31Asp2_mSS}, or mock at 0 and 3 dpi with virPst. The mean±RSE (n=12) from three independent experiments is presented. FIG. 5C shows that tRF^{31Asp2} promotes enhanced resistance to aphids. The green peach aphid (GPA) numbers at 5 dpi on WT leaves were pretreated with tRF^{31Asp2}, and tRF^{31Asp2_mSS}. Untreated and the buffer-treated WT were used as a control. Plants were initially infested with three adult apterous aphids/plant after 24 hpi. The same experiment was conducted twice with similar results. The mean±SE (n=13) is presented. FIG. 5D shows the predicted secondary structure of full-length Asp-2-tRNA^{GTC} and its 31 nt-long 5′-tRF (tRF^{31Asp2}) by RNAfold. FIG. 5E shows PR1 induction by tRF^{31Asp2} and its mutants at 24 hpi, as examined by qRT-PCR. Relative transcription levels are presented as the mean±SE (n=3). FIG. 5F shows antisense oligonucleotides or buffer were infiltrated into leaves 1 day before distal-infection with avrPst. PR1 was assessed at 1 dpi by qRT-PCR. The mean±SE was measured (n=4). Three independent experiments were performed with similar results. FIG. 5G shows PR1 induction in dcl and ago mutants at 6 hpi. Significance for indicated pairs is noted. *P<0.05; **P<0.01; ***P<0.001 (t-test except for FIG. 5C where Tukey's HSD test was used).

FIGS. 6A-6D demonstrate that tRF^{31Asp2} secondary structure is important for its biological activity. FIG. 6A shows that tRF^{31Asp2} infiltration triggers dose-dependent expression of PR1, measured by qRT-PCR. FIG. 6B provides a schematic representation of mutations targeting the primary and secondary structure of tRF^{31Asp2}. FIG. 6C shows that PR1 induction by tRF^{31Asp2} or the 3′ addition/deletion mutants was examined by qRT-PCR at 6 hpi. Relative transcription levels are presented as the mean±SE (n=3). FIG. 6D shows in vivo affinity precipitation (AP) assay of HA-AGO2 transgenic plants with biotin (Bn)-labeled tRF^{31Asp2} or tRF^{31Asp2_mSS}. Input and APed samples were subjected to IB (immunoblot) analysis using α-HA and α-streptavidin antibodies.

FIGS. 7A-7C show that tRF^{31Asp2} binds a sequence present in Gypsy retrotransposons and nearby ETI-induced genes. FIG. 7A shows induction patterns in ETI-DEGs (lower) and non-ETI-DEGs (upper). The y- and x-axes show normalized read counts from RNA-seq of tRF^{31Asp2}-treated WT and the mean values of three controls, respectively. FIG. 7B shows that the primer binding site of Gypsy retrotransposons is highly homologous to tRF^{31Asp2}. FIG. 7C shows the distance of tRF-DEGs and non-tRF-DEGs from the nearest tRF^{31Asp2}-binding ChAP-seq peak, which are presented in green and red, respectively. Distance from transposon- and non-transposon-associated tRF^{31Asp2} binding sites are shown (left and right panels, respectively). ChAP-seq peaks were further grouped based on the sequence homology score to tRF^{31Asp2} (E score). Data are represented as boxplots where the middle line and the dot are respectively the median and the mean, the lower and upper hinges correspond to the first and third quartiles, and the upper/lower whisker extends from the hinge to the largest/smallest value no further than 1.5×IQR (interquartile range). Significance for indicated pairs is noted; **P<0.01 (t-test).

FIGS. 8A-8B show that tRF^{31Asp2} physically associates with genic regions of chromatin. FIG. 8A shows that the distribution of ChAP-seq reads from regions around the TSS (transcription start sites) and TTS (transcription termination sites) of tRF-DEGs in tRF^{31Asp2}- and tRF^{31Asp2_mSS}-infiltrated WT plants. Normalized read counts per 10 million (RP10M) were estimated within each 10-bp interval, and merged from all four independent batches. The differences between tRF^{31Asp2} and tRF^{31Asp2_mSS} are presented as the top line. FIG. 8B shows read-density heatmap of indicated RNA-seq and ChAP-seq analyses for PR1, PR2, and PR5. Sites carrying a sequence homologous to tRF^{31Asp2} are denoted with a thick line

FIGS. 9A-9B illustrate that select 31 nt-long tRFs form secondary structures comparable to that of AspGTC and trigger the induction of a defense gene, PR1. FIG. 9A shows secondary structures of select tRF (31 nt long) sequences. FIG. 9B provides data related to the expression of PR1, as measured using real-time RT-PCR with PR1-specific primers relative to RHIP1, a housekeeping gene. Results are expressed as means±SE (n=5). AlaCGC used as a negative control in FIG. 9B did not display any secondary structures similar to those in FIG. 9A.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials define a term in a manner that contradicts the definition of that term in this application, this application controls.

Pathogens and pests can have a major impact on life. Biotic stress is one of the major factors leading to economically devastating diseases. Such biotic stresses can be caused by bacterial pathogens, fungal pathogens, and/or viral pathogens.

In particular, most pathogenic plant bacteria belong to the Erwinia, Pectobacterium, Pantoea, Agrobacterium, Pseudomonas, Ralstonia, Burkholderia, Acidovorax, Xanthomonas, Clavibacter, Streptomyces, Xylella, Spiroplasma, and Phytoplasma genera. There are over 200 species of pathogenic plant bacteria.

Symptoms of infected plants can be quite varied depending on the specific bacteria causing the disease. Such symptoms can include galls, overgrowths, wilts, leaf spots, specks, blights, soft rots, and scabs or cankers. Bacteria cause these symptoms through diverse specific mechanisms that include toxins or colonization of plant vasculature, and disrupting distribution of water and nutrients.

Plants resist pathogens that breach their passive defenses by detecting conserved Pathogen-Associated Molecular Patterns (PAMPs) and activating PAMP-triggered immunity (PTI). Some pathogens have evolved effectors that, following transport into host cells, facilitate evasion/suppression of PTI. The low resistance observed in plants infected with these pathogens is termed basal immunity. In turn, plants have evolved resistance (R) proteins that interact with their cognate pathogen-encoded effector(s) and induce a robust defense response termed effector-triggered immunity (ETI). Most transcripts induced during ETI also accumulate during PTI and basal immunity, albeit with weaker or delayed transcriptional dynamics.

RNA silencing is an important mechanism whereby noncoding small RNAs (sRNAs) regulate gene expression at either the post-transcriptional level, by modulating RNA stability/translatability, or at the transcriptional level, via DNA/histone modification or chromatin remodeling. The sRNAs are generated mostly by ribonuclease III-like enzymes, termed Dicer-like (DCL) proteins, in plants. Following loading onto Argonaute (AGO) proteins to form RNA-induced silencing complexes (RISC), the sRNA guide strand directs the RISC to complementary RNA targets for post-transcriptional or transcriptional gene silencing. In Arabidopsis (Arabidopsis thaliana), infection with avirulent bacterial pathogen Pseudomonas syringae pv. tomato (Pst) carrying the effector avrRpt2 (avrPst) significantly induces AGO2, which together with its homologs AGO3 and AGO7 is required for full ETI.

The sRNAs derived from tRNAs (tsRNAs) also are identified as important regulatory molecules. Several classes of tsRNAs have been identified. The tRNA-halves (˜35 nts), generated via cleavage at the anticodon loop by a ribonuclease such as angiogenin, inhibit protein synthesis by targeting translation machinery. The remaining classes, collectively termed tRNA-derived fragments (tRFs), include 5′-tRFs, 3′-tRFs, and i-tRFs. The tRFs (<˜32 nts) are generated by nucleases like Dicer and RNase Z, associate with AGOs to mediate post-transcriptional gene silencing. Dysregulation of tsRNAs is associated with disease and pathogen infection in animals. However, the role of tRFs in plant immunity is unknown.

Current methods used to help control bacterial infections in plants are varied. They include environmental controls to help prevent infection, use of specific resistant or pathogen-free varieties of plants, and use of different chemicals or agents to prevent or treat infection. Agents used to control or treat infections include copper-containing agents, biologics, antibiotics, and other chemicals. These methods can be effective, but some bacteria can develop resistance.

As such, a need exists for more effective compounds and methods for modulating stress responses in eukaryotic organisms (e.g., plants) against various sources of environmental stress. For instance, a need exists for treating or preventing the infection of eukaryotic organisms (e.g., plants) with various pathogens. Various embodiments of the present disclosure address the aforementioned needs through the utilization of RNA fragments and antisense nucleotides.

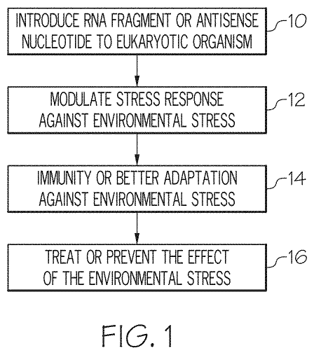

In some embodiments, the present disclosure pertains to methods of modulating a stress response in a eukaryotic organism (e.g., a plant or seed) against an environmental stress. In some embodiments illustrated in FIG. 1, the methods of the present disclosure include: introducing an RNA fragment (RF) or an antisense nucleotide to the eukaryotic organism (step 10) such that the RF or the antisense nucleotide modulates the stress response against the environmental stress (step 12) and thereby makes the eukaryotic organism immune or better adapted against the environmental stress (step 14). In some embodiments, the method is utilized to treat or prevent the effect of the environmental stress (e.g., infection or pathogenesis) in the eukaryotic organism (step 16).

Further embodiments of the present disclosure pertain to the aforementioned RFs and antisense nucleotides, which are capable of modulating a stress response in a eukaryotic organism against environmental stress. As set forth in more detail herein, the present disclosure can have numerous embodiments. In particular, the present disclosure can utilize various RFs and antisense nucleotides in order to modulate stress responses in various eukaryotic organisms against various environmental stresses through various mechanisms of action.

### RNA Fragments (RFs)

In some embodiments, an RF may be introduced to a eukaryotic organism. The present disclosure can utilize various RFs. For instance, in some embodiments, the RFs include a sequence recognition site and a stem loop structure. In some embodiments, the sequence recognition site includes a reverse complement sequence of a nucleotide sequence of the organism. In some embodiments, the sequence recognition site is in single-stranded form.

In some embodiments, the stem loop structure of the RF includes a paired region that includes paired RNA nucleotides. In some embodiments, the stem loop structure also includes an unpaired region that includes unpaired RNA nucleotides in the form of a loop. In some embodiments, the RF may be in isolated form.

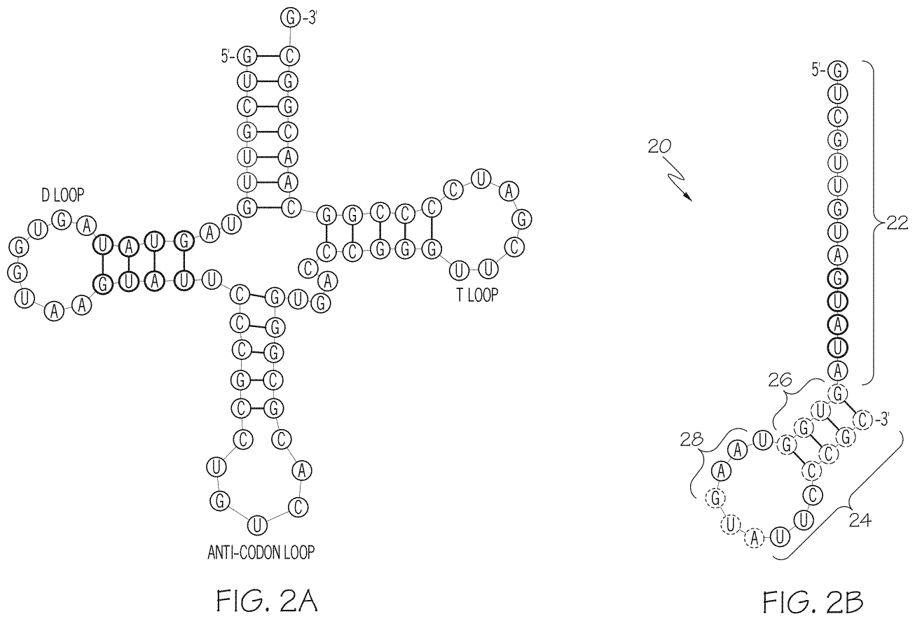

In some embodiments, the RFs of the present disclosure have the structure illustrated as RF 20 in FIG. 2B, which may be derived from tRNAs illustrated in FIG. 2A. In the embodiment illustrated in FIG. 2B, RF 20 includes sequence recognition site 22, and stem loop structure 24, which includes paired region 26 and unpaired region 28.

In some embodiments, the RFs of the present disclosure include transfer RNA (tRNA)-derived RNA fragments (tRFs). In some embodiments, the tRFs of the present disclosure are derived from tRNAs that include, without limitation, tRNA^{Asp}, tRNA^{Gly}, tRNA^{Glu}, tRNA^{His}, and combinations thereof. In some embodiments, the tRFs of the present disclosure are derived from tRNA^{Asp}. In some embodiments, the tRFs of the present disclosure are derived from tRNA^{Asp(GTC)}.

In some embodiments, the tRFs of the present disclosure lack a tRNA anti-codon loop. In some embodiments, the tRFs of the present disclosure lack a tRNA T-loop. In some embodiments, the tRFs of the present disclosure lack a tRNA acceptor stem. In some embodiments, the tRFs of the present disclosure lack a tRNA variable loop. In some embodiments, the tRFs of the present disclosure lack a tRNA anti-codon loop, T-loop, acceptor stem and variable loop.

In some embodiments, the tRFs of the present disclosure include a D-loop portion of tRNAs. In some embodiments, the tRFs of the present disclosure only include a D-loop portion of tRNAs.

The RFs of the present disclosure can have various sequences. For instance, in some embodiments, the RF includes, without limitation, at least one of 5′ GUCGUUGUAGUAUAGUGGUAAGUAUUCCCGC-3′ (SEQ ID NO: 1), 5′-UCCGUUGUAGUCUAGCUGGUCAGGAUACUCG-3′ (SEQ ID NO: 2), 5′-UCCGAUGUCGUCCAGCGGUUAGGAUAUCUGG-3′ (SEQ ID NO: 3), 5′-GCGUCUGUAGUCCAACGGUUAGGAUAAUUGC-3′ (SEQ ID NO: 4), or a sequence with at least 75% identity with any one of SEQ ID NOS: 1-4.

In some embodiments, the RF sequence includes 5′-GUCGUUGUAGUAUAGUGGUAAGUAUUCCCGC-3′ (SEQ ID NO: 1). In some embodiments, 5′-GUCGUUGUAGUAUA-3′ in SEQ ID NO: 1 represents the sequence recognition site. In some embodiments, 5′-GUGGUAAGUAUUCCCGC-3′ in SEQ ID NO: 1 represents the stem loop structure. In some embodiments, 5′-GUGG-3′ in SEQ ID NO: 1 pairs with 5′-CCGC-3′ in SEQ ID NO: 1 to form the paired region of the stem loop structure. In some embodiments, 5′-UAAGUAUUC-3′ in SEQ ID NO: 1 represents the loop of the stem loop structure.

In some embodiments, the RF sequence includes a sequence with at least 65% identity to SEQ ID NO: 1. In some embodiments, the RF sequence includes a sequence with at least 70% identity to SEQ ID NO: 1. In some embodiments, the RF sequence includes a sequence with at least 75% identity to SEQ ID NO: 1. In some embodiments, the RF sequence includes a sequence with at least 80% identity to SEQ ID NO: 1. In some embodiments, the RF sequence includes a sequence with at least 85% identity to SEQ ID NO: 1. In some embodiments, the RF sequence includes a sequence with at least 90% identity to SEQ ID NO: 1. In some embodiments, the RF sequence includes a sequence with at least 95% identity to SEQ ID NO: 1. In some embodiments, the RF sequence includes a sequence with at least 99% identity to SEQ ID NO: 1.

In some embodiments, the RF sequence includes 5′-UCCGUUGUAGUCUAGCUGGUCAGGAUACUCG-3′ (SEQ ID NO: 2). In some embodiments, 5′-UCCGUUGUAGUCUAGC-3′ in SEQ ID NO: 2 represents the sequence recognition site. In some embodiments, 5′-UGGUCAGGAUACUCG-3′ in SEQ ID NO: 2 represents the stem loop structure. In some embodiments, 5′-UGG-3′ in SEQ ID NO: 2 pairs with 5′-UCG-3′ in SEQ ID NO: 2 to form the paired region of the stem loop structure. In some embodiments, 5′-UCAGGAUAC-3′ in SEQ ID NO: 2 represents the loop of the stem loop structure.

In some embodiments, the RF sequence includes a sequence with at least 65% identity to SEQ ID NO: 2. In some embodiments, the RF sequence includes a sequence with at least 70% identity to SEQ ID NO: 2. In some embodiments, the RF sequence includes a sequence with at least 75% identity to SEQ ID NO: 2. In some embodiments, the RF sequence includes a sequence with at least 80% identity to SEQ ID NO: 2. In some embodiments, the RF sequence includes a sequence with at least 85% identity to SEQ ID NO: 2. In some embodiments, the RF sequence includes a sequence with at least 90% identity to SEQ ID NO: 2. In some embodiments, the RF sequence includes a sequence with at least 95% identity to SEQ ID NO: 2. In some embodiments, the RF sequence includes a sequence with at least 99% identity to SEQ ID NO: 2.

In some embodiments, the RF sequence includes 5′-UCCGAUGUCGUCCAGCGGUUAGGAUAUCUGG-3′ (SEQ ID NO: 3). In some embodiments, 5′-UCCGAUGUCGUCCAGCGG-3′ in SEQ ID NO: 3 represents the sequence recognition site. In some embodiments, 5′-UUAGGAUAUCUGG-3′ in SEQ ID NO: 3 represents the stem loop structure. In some embodiments, 5′-UUAG-3′ in SEQ ID NO: 3 pairs with 5′-CUGG-3′ in SEQ ID NO: 3 to form the paired region of the stem loop structure. In some embodiments, 5′-GAUAU-3′ in SEQ ID NO: 3 represents the loop of the stem loop structure.

In some embodiments, the RF sequence includes a sequence with at least 65% identity to SEQ ID NO: 3. In some embodiments, the RF sequence includes a sequence with at least 70% identity to SEQ ID NO: 3. In some embodiments, the RF sequence includes a sequence with at least 75% identity to SEQ ID NO: 3. In some embodiments, the RF sequence includes a sequence with at least 80% identity to SEQ ID NO: 3. In some embodiments, the RF sequence includes a sequence with at least 85% identity to SEQ ID NO: 3. In some embodiments, the RF sequence includes a sequence with at least 90% identity to SEQ ID NO: 3. In some embodiments, the RF sequence includes a sequence with at least 95% identity to SEQ ID NO: 3. In some embodiments, the RF sequence includes a sequence with at least 99% identity to SEQ ID NO: 3.

In some embodiments, the RF sequence includes 5′-GCGUCUGUAGUCCAACGGUUAGGAUAAUUGC-3′ (SEQ ID NO: 4). In some embodiments, 5′-GCGUCUGUAGUCCAA-3′ in SEQ ID NO: 4 represents the sequence recognition site. In some embodiments, 5′-CGGUUAGGAUAAUUG-3′ in SEQ ID NO: 4 represents the stem loop structure. In some embodiments, 5′-CGGUUA-3′ in SEQ ID NO: 4 pairs with 5′-UAAUUG-3′ in SEQ ID NO: 4 to form the paired region of the stem loop structure. In some embodiments, 5′-GGA-3′ in SEQ ID NO: 4 represents the loop of the stem loop structure.

In some embodiments, the RF sequence includes a sequence with at least 65% identity to SEQ ID NO: 4. In some embodiments, the RF sequence includes a sequence with at least 70% identity to SEQ ID NO: 4. In some embodiments, the RF sequence includes a sequence with at least 75% identity to SEQ ID NO: 4. In some embodiments, the RF sequence includes a sequence with at least 80% identity to SEQ ID NO: 4. In some embodiments, the RF sequence includes a sequence with at least 85% identity to SEQ ID NO: 4. In some embodiments, the RF sequence includes a sequence with at least 90% identity to SEQ ID NO: 4. In some embodiments, the RF sequence includes a sequence with at least 95% identity to SEQ ID NO: 4. In some embodiments, the RF sequence includes a sequence with at least 99% identity to SEQ ID NO: 4.

The RFs of the present disclosure can include various sequence recognition sites. For instance, in some embodiments, the sequence recognition sites include a reverse complement sequence of a nucleotide sequence of an organism. In some embodiments, the nucleotide sequence includes a DNA sequence. In some embodiments, the nucleotide sequence includes an RNA sequence, such as an mRNA sequence.

The RFs of the present disclosure can have various lengths. For instance, in some embodiments, RFs of the present disclosure have a nucleotide length of at least 30 bases. In some embodiments, RFs of the present disclosure have a nucleotide length of at least 31 bases.

In some embodiments, the RF sequence recognition site include at least 10 bases. In some embodiments, the RF sequence recognition site include at least 14 bases. In some embodiments, the RF paired region of the stem loop structure includes at least 3 base pairs. In some embodiments, the RF unpaired region of the stem loop structure includes at least 3 bases.

### Antisense Nucleotides

In some embodiments, an antisense nucleotide may be introduced to a eukaryotic organism. In some embodiments, the antisense nucleotide is an RNA or a DNA nucleotide. In some embodiments, the antisense nucleotide is in isolated form.

In some embodiments, the antisense nucleotide includes a reverse complement sequence of SEQ ID NO: 1. In some embodiments, the antisense nucleotide includes 5′-GCGGGAAUACUUACCACUAUACUACAACGAC-3′ (SEQ ID NO: 5). In some embodiments, the antisense nucleotide includes 5′-GCGGGAATACTTACCACTATACTACAACGAC-3′ (SEQ ID NO: 6). In some embodiments, the antisense nucleotide includes at least an antisense nucleotide for a sequence recognition site of SEQ ID NO: 1. In some embodiments, the antisense nucleotide includes 5′-GTCGTTGTAGTAT-3′ (SEQ ID NO: 7). In some embodiments, the antisense nucleotide includes 5′-GUCGUUGUAGUAU-3′ (SEQ ID NO: 8).

In some embodiments, the antisense nucleotide includes a reverse complement sequence of SEQ ID NO: 2. In some embodiments, the antisense nucleotide includes 5′-CGAGUAUCCUGACCAGCUAGACUACAACGGA-3′ (SEQ ID NO: 9). In some embodiments, the antisense nucleotide includes 5′-CGAGTATCCTGACCAGCTAGACTACAACGGA-3′ (SEQ ID NO: 10). In some embodiments, the antisense nucleotide includes at least an antisense nucleotide for a sequence recognition site of SEQ ID NO: 2. In some embodiments, the antisense nucleotide includes 5′-GCUAGACUACAACGGA-3′ (SEQ ID NO: 11). In some embodiments, the antisense nucleotide includes 5′-GCTAGACTACAACGGA-3′ (SEQ ID NO: 12).

In some embodiments, the antisense nucleotide includes a reverse complement sequence of SEQ ID NO: 3. In some embodiments, the antisense nucleotide includes 5′-CCAGAUAUCCUAACCGCUGGACGACAUCGGA-3′ (SEQ ID NO: 13). In some embodiments, the antisense nucleotide includes 5′-CCAGATATCCTAACCGCTGGACGACATCGGA-3′ (SEQ ID NO: 14). In some embodiments, the antisense nucleotide includes at least an antisense nucleotide for a sequence recognition site of SEQ ID NO 3. In some embodiments, the antisense nucleotide includes 5′-CCGCUGGACGACAUCGGA-3′ (SEQ ID NO: 15). In some embodiments, the antisense nucleotide includes 5′-CCGCTGGACGACATCGGA-3′ (SEQ ID NO: 16).

In some embodiments, the antisense nucleotide includes a reverse complement sequence of SEQ ID NO: 4. In some embodiments, the antisense nucleotide includes 5′-GCAAUUAUCCUAACCGUUGGACUACAGACGC-3′ (SEQ ID NO: 17). In some embodiments, the antisense nucleotide includes 5′-GCAATTATCCTAACCGTTGGACTACAGACGC-3′ (SEQ ID NO: 18). In some embodiments, the antisense nucleotide includes at least an antisense nucleotide for a sequence recognition site of SEQ ID NO: 4. In some embodiments, the antisense nucleotide includes 5′-UUGGACUACAGACGC-3′ (SEQ ID NO: 19). In some embodiments, the antisense nucleotide includes 5′-TTGGACTACAGACGC-3′ (SEQ ID NO: 20).

In some embodiments, the antisense nucleotide includes a sequence with at least 65% identity with any one of SEQ ID NOS: 5-20. In some embodiments, the antisense nucleotide includes a sequence with at least 75% identity with any one of SEQ ID NOS: 5-20. In some embodiments, the antisense nucleotide includes a sequence with at least 80% identity with any one of SEQ ID NOS: 5-20. In some embodiments, the antisense nucleotide includes a sequence with at least 85% identity with any one of SEQ ID NOS: 5-20. In some embodiments, the antisense nucleotide includes a sequence with at least 90% identity with any one of SEQ ID NOS: 5-20. In some embodiments, the antisense nucleotide includes a sequence with at least 95% identity with any one of SEQ ID NOS: 5-20. In some embodiments, the antisense nucleotide includes a sequence with at least 99% identity with any one of SEQ ID NOS: 5-20.

In some embodiments, the reverse complement sequence of the sequence recognition site of the RF or the antisense nucleotide binds to a retrotransposon region of the organism. In some embodiments, the retrotransposon region includes a Gypsy superfamily of long-terminal repeat (LTR)-retrotransposons. In some embodiments, the retrotransposon region includes a Copia superfamily of long-terminal repeat (LTR)-retrotransposons. In some embodiments, the retrotransposon region includes a Gypsy and a Copia superfamily of long-terminal repeat (LTR)-retrotransposons. In some embodiments, the reverse complement sequence of the sequence recognition site of the RF or the antisense nucleotide binds to a genic region of an organism's chromatin. In some embodiments, the reverse complement sequence of the sequence recognition site of the RF or the antisense nucleotide binds to an intergenic region of an organism's chromatin.

### Eukaryotic Organisms

The RFs and antisense nucleotides of the present disclosure may be applied to various eukaryotic organisms. For instance, in some embodiments, the eukaryotic organisms include, without limitation, plants, seeds, and combinations thereof.

In some embodiments, the eukaryotic organisms include plants or seeds. In some embodiments, the plants or seeds include dicots. In some embodiments, the plants or seeds include monocots. In some embodiments, the plants or seeds are dicots that include, without limitation, soybean, lettuce, tomato, potato, legumes, peas, beans, lentils, peanuts, cotton, Arabidopsis, and combinations thereof.

In some embodiments, the plants or seeds are tomato plants. In some embodiments, the plants or seeds include Arabidopsis thaliana plants or seeds.

### Introduction of RFs and Antisense Nucleotides to Eukaryotic Organisms

Various methods may be utilized to introduce RFs and antisense nucleotides into eukaryotic organisms. For instance, in some embodiments, the introduction occurs by methods that include, without limitation, transfection, incubation, transferred DNA insertion, syringe injection, enhancer trap insertion, floral-dip transformation, callus transformation, tissue transformation, mobile genetic elements insertion, and combinations thereof.

The RFs and antisense nucleotides of the present disclosure may be introduced to eukaryotic organisms at various stages. For instance, in some embodiments, the introduction occurs at a seedling stage of a plant. In some embodiments, the introduction occurs at an adult stage of the plant. In some embodiments, the introduction occurs to seeds of a plant. In some embodiments, the introduction occurs at various stages of an infection in an animal, such as a human being.

### Modulation of Stress Responses Against Environmental Stress

The RFs and antisense nucleotides of the present disclosure may modulate stress responses against various sources of environmental stress. For instance, in some embodiments, the environmental stress includes, without limitation, abiotic stress, biotic stress, pathogenesis, pest infestation, and combinations thereof.

In some embodiments, the environmental stress includes pathogenesis. In some embodiments, the pathogenesis is caused by at least one pathogen. In some embodiments, the at least one pathogen includes, without limitation, a fungal pathogen, a bacterial pathogen, a viral pathogen, and combinations thereof.

In some embodiments, the environmental stress includes a pest infestation. In some embodiments, the pest infestation is caused by at least one pest. In some embodiments, the pest includes, without limitation, an aphid, a nematode, and combinations thereof.

In some embodiments, the environmental stress includes pathogenesis caused by at least one bacterial pathogen. In some embodiments, the at least one bacterial pathogen belongs to genera that include, without limitation, Erwinia, Pectobacterium, Pantoea, Agrobacterium, Pseudomonas, Ralstonia, Burkholderia, Acidovorax, Xanthomonas, Clavibacter, Streptomyces, Xylella, Spiroplasma, Phytoplasma, and combinations thereof. In some embodiments, the bacterial pathogens include pathogens that belong to the Pseudomonas genera.

Without being bound by theory, the RFs and antisense nucleotides of the present disclosure may modulate stress responses against environmental stresses (e.g., pathogens and/or pests) in various manners. For instance, in some embodiments, the RFs and antisense nucleotides of the present disclosure modulate a stress response by increasing the stress response against the environmental stress, decreasing the stress response against the environmental stress, or combinations thereof. In some embodiments, the RFs and antisense nucleotides of the present disclosure modulate a stress response by increasing the stress response against the environmental stress. In some embodiments, the RFs and antisense nucleotides of the present disclosure modulate the stress response by decreasing the stress response against the environmental stress.

In some embodiments, the stress response is an immune response. As such, in some embodiments, the RFs and antisense nucleotides of the present disclosure modulate an immune response against the environmental stress (e.g., pathogens and/or pests). In some embodiments, the RFs and antisense nucleotides of the present disclosure modulate an effector-triggered immunity (ETI) against the environmental stress (e.g., pathogens and/or pests).

Without being bound by further theory, the RFs and antisense nucleotides of the present disclosure may modulate stress responses against environmental stresses through various mechanisms of action. For instance, in some embodiments, the RFs and antisense nucleotides of the present disclosure modulate the stress response by inducing the expression of one or more defense genes. In some embodiments, the RFs and antisense nucleotides of the present disclosure modulate the stress response by initiating a specific interaction with chromatin in a sequence-specific manner. In some embodiments, the interaction results in the expression of one or more defense genes.

In some embodiments, the RFs and antisense nucleotides of the present disclosure interact with LTR (Long Terminal Repeats)-retrotransposons on chromatin as their replication and propagation requires a tRNA. In animals and plants, LTR (Long Terminal Repeats)-retrotransposons make up to 75% of nuclear DNA.

In some embodiments, the interaction of RFs and antisense nucleotides with chromatin results in the expression of various defense genes in eukaryotic organisms that in turn modulate a stress response. In some embodiments, a stress response is associated with the induction of the defense genes. In some embodiments, the defense genes include, without limitation, pathogenesis-related protein-1 (PR-1), Cyclic NUCLEOTIDE GATED CHANNEL 10 (CNGC10), GLUTAREDOXIN 13 (GRXS13), MAP KINASE 11 (MPK11), PLANT U-BOX 54 (PUB54), ACTIVATED DISEASE RESISTANCE 1 (ADR1), MYB DOMAIN PROTEIN 15 (MYB15), ACC synthase 6 (ACS6), Arabidopsis BAX INHIBITOR 1 (ATBI-1), Ca²⁺-DEPENDENT MODULATOR OF ICR1 (CMI1), CCR4-ASSOCIATED FACTOR 1A (AtCAF1a), DICARBOXYLATE CARRIER 2 (DIC2), ETHYLENE RESPONSE FACTOR 104 (ERF104), NDR1/HIN1-LIKE 3 (NHL3), NUDIX HYDROLASE HOMOLOG 21 (NUDT21), SALT TOLERANCE ZINC FINGER (STZ), THIOREDOXIN H-TYPE 5 (ATHS), CALMODULIN-LIKE 24 (CML24), analogs thereof, homologs thereof, and combinations thereof. In some embodiments, the defense genes include PR-1, analogs thereof, homologs thereof, and combinations thereof.

In some embodiments, the defense genes include analogs of any of the aforementioned genes. In some embodiments, the analogs share at least 95% sequence identity with any of the aforementioned genes. In some embodiments, the analogs share at least 90% sequence identity with any of the aforementioned genes. In some embodiments, the analogs share at least 85% sequence identity with any of the aforementioned genes. In some embodiments, the analogs share at least 80% sequence identity with any of the aforementioned genes. In some embodiments, the analogs share at least 75% sequence identity with any of the aforementioned genes. In some embodiments, the analogs share at least 70% sequence identity with any of the aforementioned genes.

In some embodiments, the defense genes include homologs of any of the aforementioned genes. In some embodiments, the homologs share at least 95% sequence identity with any of the aforementioned genes. In some embodiments, the homologs share at least 90% sequence identity with any of the aforementioned genes. In some embodiments, the homologs share at least 85% sequence identity with any of the aforementioned genes. In some embodiments, the homologs share at least 80% sequence identity with any of the aforementioned genes. In some embodiments, the homologs share at least 75% sequence identity with any of the aforementioned genes. In some embodiments, the homologs share at least 70% sequence identity with any of the aforementioned genes.

### Applications and Advantages

The RFs, antisense nucleotides and methods of the present disclosure can have various advantages. For instance, in some embodiments, the RFs and antisense nucleotides of the present disclosure help numerous eukaryotic organisms (e.g., plants and seeds) develop immunity against various environmental stresses by harnessing the eukaryotic organism's innate biological processes. In some embodiments, the RFs and antisense nucleotides of the present disclosure help numerous eukaryotic organisms (e.g., plants and seeds) to modulate stress responses against various environmental stresses by enhancing or reducing the eukaryotic organism's stress responses.

As such, the RFs, antisense nucleotides, and methods of the present disclosure can have numerous applications. For instance, in some embodiments, the RFs, antisense nucleotides, and methods of the present disclosure may be utilized to enhance the yields of various crops by protecting plants and seeds from numerous pathogens and/or pests. In some embodiments, the RFs, antisense nucleotides, and methods of the present disclosure may be utilized to improve the health of plants by enhancing or reducing stress responses.

### Additional Embodiments

Reference will now be made to more specific embodiments of the present disclosure and experimental results that provide support for such embodiments. However, Applicant notes that the disclosure herein is for illustrative purposes only and is not intended to limit the scope of the claimed subject matter in any way.

### EXAMPLE 1

### A Nuclear tRNA-Derived Fragment Triggers Immunity in Arabidopsis

In Arabidopsis, effector-triggered immunity (ETI) to avirulent Pseudomonas syringae pv tomato (Pst) was found to correlate with rapid nuclear accumulation of a 31 nt 5′-tRNA fragment from Asp-tRNA (tRF^{31Asp2}) in a Dicer-Like 1 (DCL1)-dependent manner. Infiltration of Arabidopsis leaves with tRF^{31Asp2} induced >500 defense-associated genes and immunity against virulent Pst and aphids. Based on mutational analysis, the predicted stem-loop structure and 5′ sequence of tRF^{31Asp2} are important for biological activity. Chromatin affinity precipitation-sequencing revealed that tRF^{31Asp2} binds a specific sequence present in defense genes and the Gypsy superfamily of LTR (long-terminal repeats)-retrotransposons. Since the proliferation of Gypsy retrotransposons is primed by tRNA binding to this site, known as the primer binding site, a similar mechanism may allow tRF^{31Asp2} to modulate ETI-associated transcriptional reprogramming.

### EXAMPLE 1.1

### DCL1, a Positive ETI Regulator

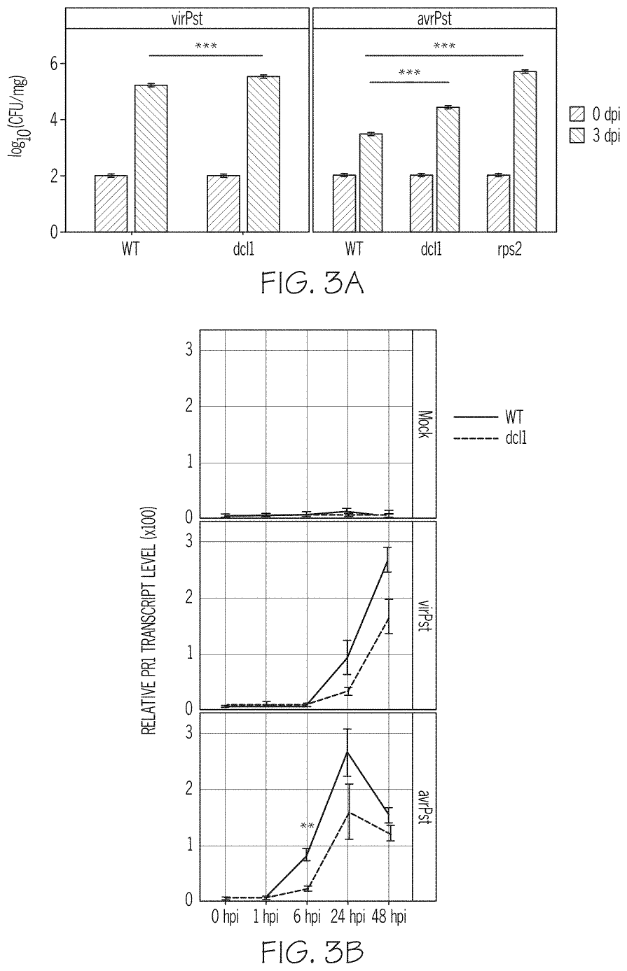

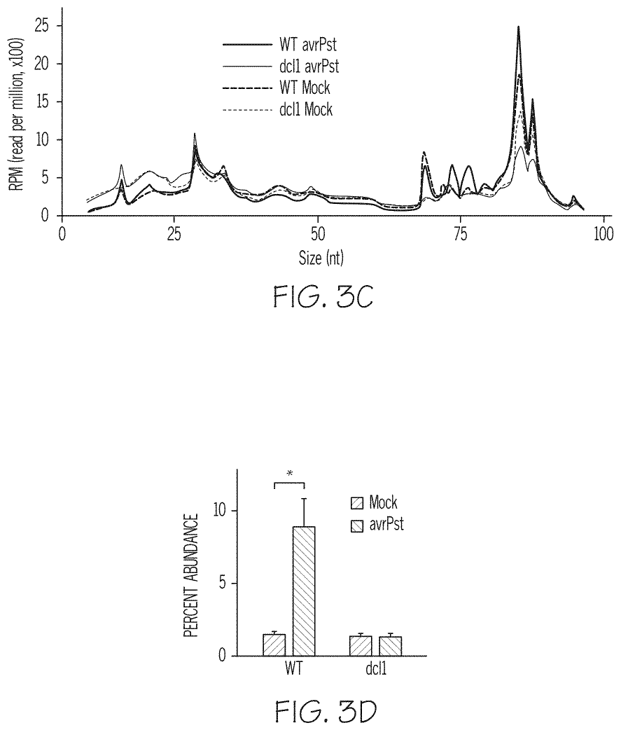

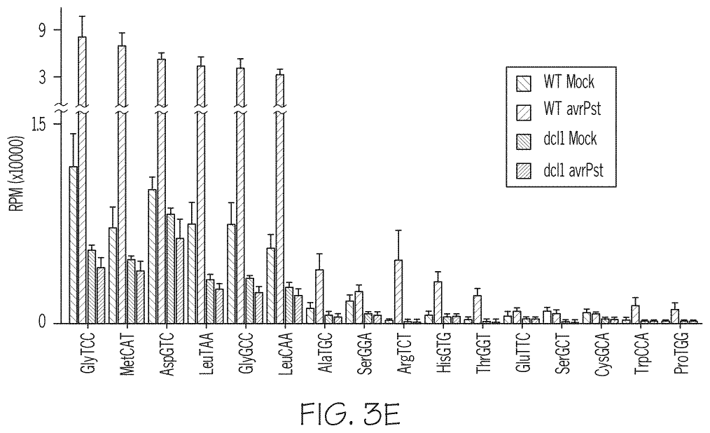

Applicant previously showed that some Arabidopsis ago and dcl mutants are compromised for basal resistance to virulent Pst (virPst) and/or ETI to avrPst. To confirm that loss of DCL1 compromised ETI without substantially affecting basal resistance, bacterial growth and expression of defense gene Pathogenesis-related 1 (PRI) were monitored in dcl1-7 and wild type (WT). dcl1-7 supported significantly higher levels of avrPst than WT at 3 day-post-inoculation (dpi), whereas growth of virPst in these plants was marginally higher (FIG. 3A). PR1 expression was significantly dampened in avrPst-inoculated dcl1-7 vs WT at 6 hour-post-inoculation (hpi). However, the expression was marginally different at later points, or at any timepoint after virPst inoculation (FIG. 3B).

Thus, without being bound by theory, DCL1 may play an early role in the induction of defense responses. To investigate whether DCL1 modulates transcriptional reprogramming following pathogen infection, the transcriptomes of mock-, avrPst- and virPst-inoculated Arabidopsis were analyzed. Pair-wise comparisons revealed that the number of differentially expressed genes (DEGs) in mock vs avrPst and avrPst vs virPst peaked at 6 hpi, whereas those in mock vs virPst peaked at 24 hpi. Most of the upregulated DEGs in avrPst vs virPst (ETI-DEGs) at 6 hpi displayed little overlap with those at other timepoints. Since early ETI-associated responses occur within minutes of inoculation, the machinery responsible for ETI-DEG induction presumably functions between these key ETI events.

Whether DCL1-associated sRNAs regulate ETI-induced transcriptional reprogramming was assessed by characterizing nuclear-localized sRNAs (<100 nt) in mock- and avrPst-inoculated WT and dcl1-7 at 1 hpi. Size-distribution analysis showed that, regardless of pathogen inoculation, dcl1-7 accumulated more RNAs of ≤28 nt, whereas WT accumulated more RNAs of ≥70 nt (FIG. 3C). Additionally, while the nuclear RNA distribution profile was not substantially altered in dcl1-7 after avrPst inoculation, a ˜2-fold increase in 74 nt and 76/77 nt RNAs was observed in pathogen- vs mock-inoculated WT; a significant increase also was observed in 85/86 nt. Examining >70 nt RNA revealed that many originated from tRNA genes. Full-length Arabidopsis tRNAs are 71 to 92 nt. The tRNA reads in the nuclei of avrPst-inoculated WT, but not dcl1-7, rose to nearly 10% of the total sRNA population by 1 hpi (FIG. 3D). Dramatic pathogen-induced, DCL1-dependent increases were observed for a subset of tRNAs (FIG. 3E).

### EXAMPLE 1.2.

### Early Rise of DCL1-Dependent tRFs in ETI

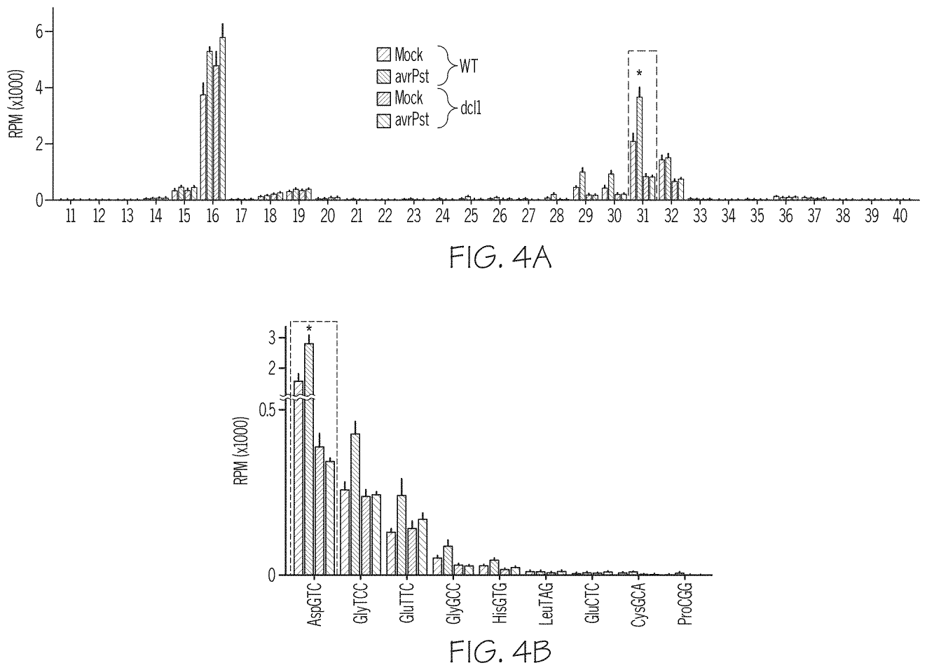

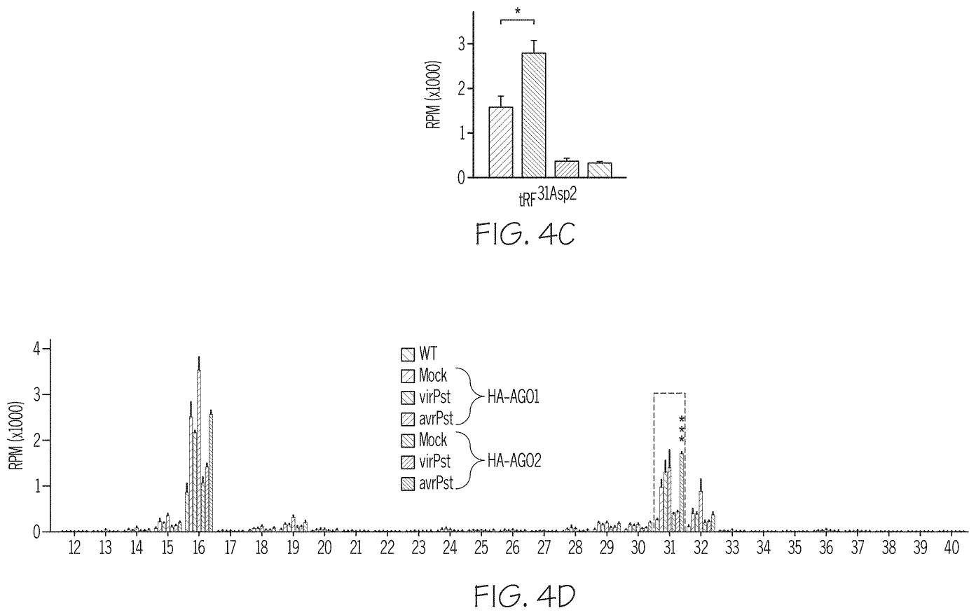

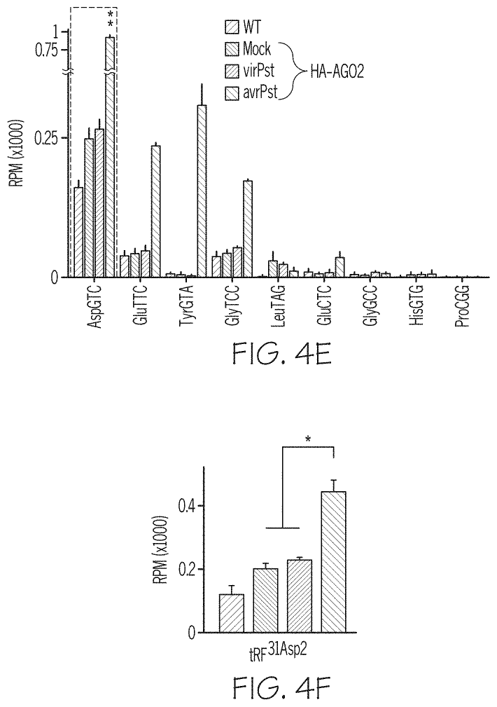

Since nuclear tRNA levels rose during ETI, Applicant assessed whether nuclear sRNAs derived from tRNAs also increased. Due to tRNA gene redundancy, a sequence-centered analysis, termed MINTmap, Applicant used to characterize nuclear sRNAs derived from tRNAs of 11 to 40 nts in mock- and avrPst-inoculated WT and dcl1-7 at 1 hpi. Of the four classes of sRNAs derived from tRNAs, 5′-tRFs and 5′-halves predominated. Of these, only 5′-tRFs were induced by avrPst in a DCL1-dependent manner. Size distribution analysis of 5′-tRFs revealed peaks at 16 and 31 nt, with the 31 nt tRFs exhibiting DCL1-dependent accumulation after avrPst inoculation (FIG. 4A).

The 31 nt tRFs were largely derived from a few tRNAs, with AspGTC predominating (FIG. 4B). The pathogen- and DCL1-dependent accumulation of a 31 nt 5′-tRF derived from Asp-2-tRNA^{GTC} (one of the genes encoding tRNA^{Asp}; designated tRF^{31Asp2}) is illustrated in FIG. 4C. This tRF was largely responsible for the increase in AspGTC.

Since Argonautes (AGOs) are effector proteins for some tRFs and tRNA-halves, the possibility that they interact with nuclear-localized tRFs was assessed. Of the ten Arabidopsis AGOs, AGO1 and AGO2 were selected as they are the most highly expressed in mature leaves, and AGO2 was implicated in ETI. Additionally, marginal increases in nuclear-localized AGO1 and AGO2 were noted following pathogen inoculation.

Analysis of the 5′-tRF profile from nuclear-localized AGO1- and AGO2-bound sRNAs in mock-, avrPst- and virPst-inoculated WT at 1 hpi revealed major peaks at 16 and 31 nt (FIG. 4D). Infection with avrPst, but not virPst, substantially increased the abundance of AGO1/2-bound 16 nt tRFs and AGO2-bound 31 nt tRFs. Similar to the nuclear tRFs (FIG. 4B), AGO2-bound 31 nt tRFs were derived from a subset of tRNAs, with AspGTC predominating (FIG. 4E). Likewise, avrPst-induced accumulation of AspGTC was largely due to increases in tRF^{31Asp2} (FIG. 4F). Together, these findings suggest that ETI correlates with the DCL1-dependent accumulation of AGO2-associated tRF^{31Asp2} in the nucleus.

### EXAMPLE 1.3

### tRF-Triggered Immunity

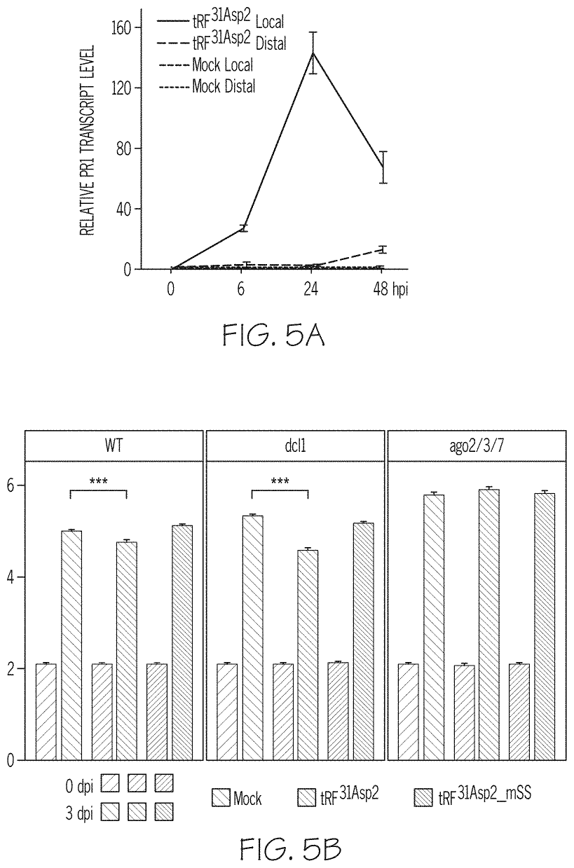

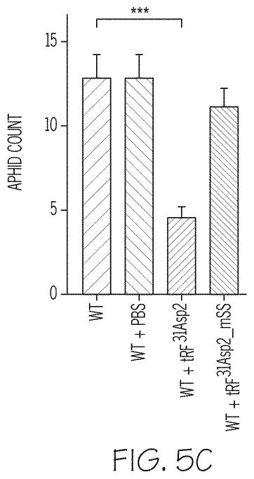

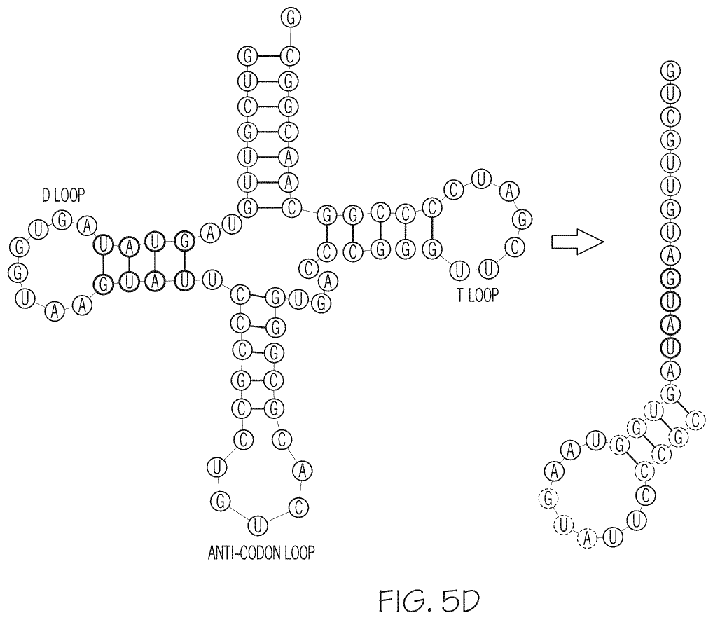

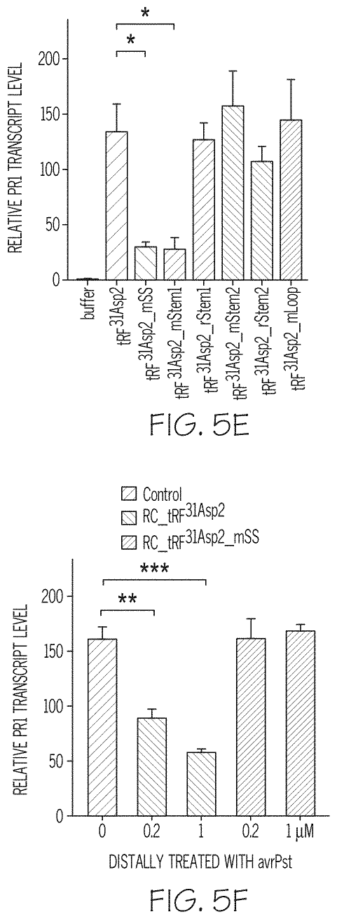

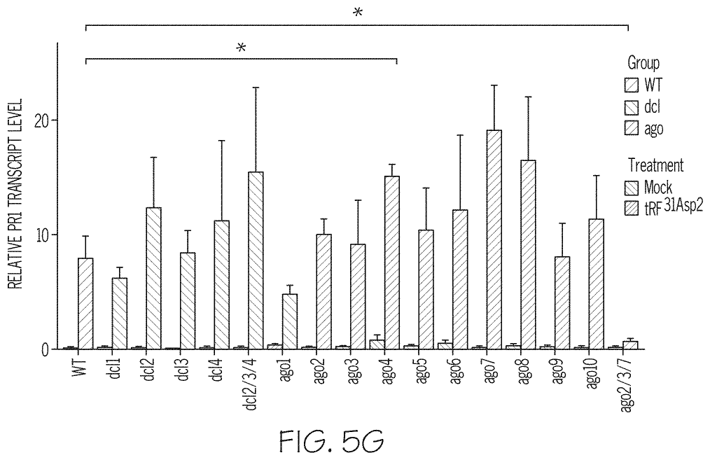

To investigate the role of tRF^{31Asp2} in defense responses, synthetic tRF^{31Asp2} RNA was infiltrated into Arabidopsis leaves. The tRF^{31Asp2} induced PR1 expression in a dose-dependent manner. A time course revealed that PR1 expression in infiltrated leaves peaked at 24 hpi, and an increase in systemic, untreated leaves was observed at 48 hpi (FIG. 5A). Pretreating WT with tRF^{31Asp2} vs mock led to a ˜43% reduction in virPst growth at 3 dpi (FIG. 5B). An even greater reduction was observed in tRF^{31Asp2}-pretreated dcl1-7, suggesting that DCL1 might regulate tRF^{31Asp2} degradation as well as synthesis. Alternatively, defense-related DCL1-associated sRNAs, such as miR393, might interact with tRF^{31Asp2}, although an underlying mechanism remains to be characterized.

To expand the analysis, the ability of tRF^{31Asp2} to induce aphid resistance was also tested. Aphid resistance was enhanced significantly in WT pretreated with tRF^{31Asp2} but not buffer control (FIG. 5C). Together, these findings indicate that tRF^{31Asp2} infiltration induces immune responses.

### EXAMPLE 1.4

### A Stem-Loop in the tRF Structure

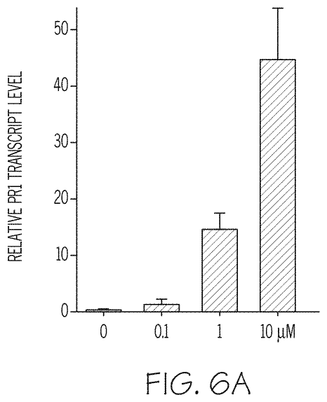

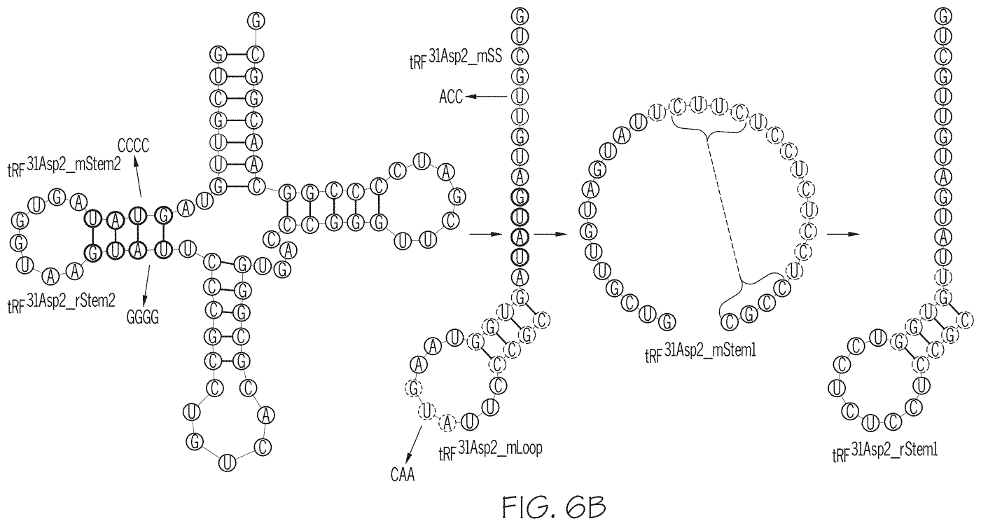

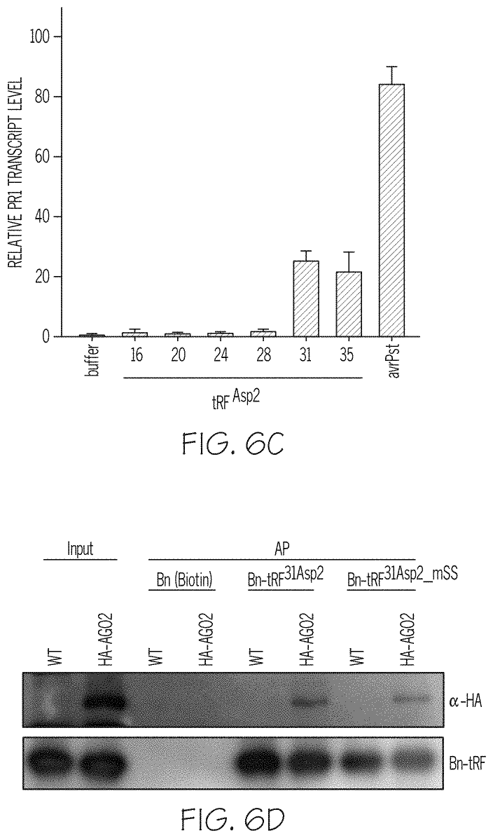

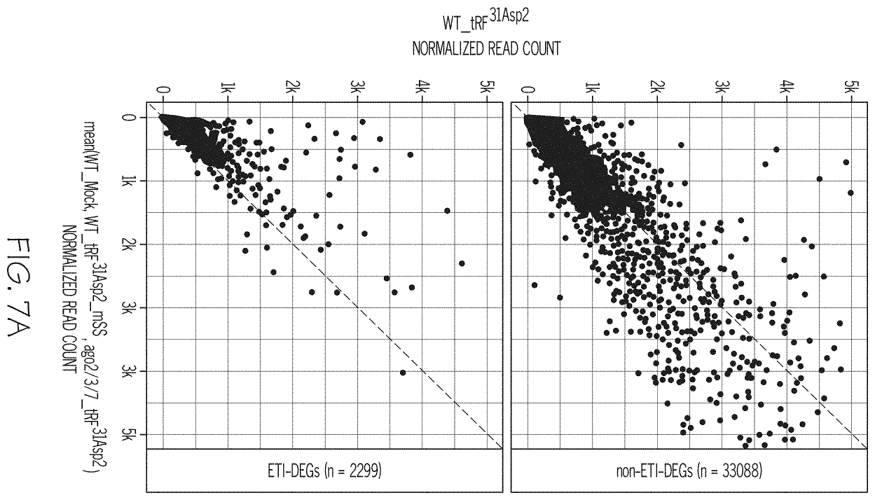

The tRF^{31Asp2} has a predicted secondary structure with a stem-loop whose location differs from the tRNA D arm (FIG. 5D). To characterize which regions of tRF^{31Asp2} are important for biological activity, synthetic RNAs bearing mutations at four sites were generated (FIG. 6B). Additionally, the effect of deleting/adding nts to the 3′ end of tRF^{31Asp2}, based on the sequence of its precursor Asp-2-tRNA^{GTC}, was assessed (FIG. 6C). The tRF^{31Asp2_mSS} with three altered nts in the 5′ single-strand region (FIG. 5C) was substantially less effective at inducing PR1 expression (FIG. 5E) and immunity against virPst and aphids (FIGS. 5B and 5C). The decreased biological activity did not correlate with reduced AGO2 binding, which was comparable in tRF^{31Asp2_mSS} and tRF^{31Asp2} (FIG. 6D). The tRF^{31Asp2_mStem1}, with mutations disrupting the predicted stem structure, also was compromised for PR1 induction (FIG. 5E). However, tRF^{31Asp2_rStem1}, containing compensatory mutations restoring the stem structure, induced PR1 to comparable levels as tRF^{31Asp2} (FIG. 5E). Mutations in the predicted loop (tRF^{31Asp2_mLoop}) and alterations in tRNA D-loop-associated nts (tRF^{31Asp2_mStem2}) did not substantially alter PR1 induction (FIG. 5E). Adding 4 nts to the 3′ end of tRF^{31Asp2} also did not significantly affect PR1 induction, whereas trimming 3 or more nts from this end, which impacts the stem-loop structure, abolished PR1 induction (FIG. 6C). Whether an oligonucleotide containing the antisense sequence of tRF^{31Asp2} (RC_tRF^{31Asp2}) could suppress avrPst-induced PR1 expression was assessed. Pretreating leaves with RC_tRF^{31Asp2} prior to infecting lower leaves with avrPst suppressed systemic PR1 expression, whereas RC_tRF^{31Asp2_mSS}, the antisense sequence of tRF^{31Asp2_mSS}, did not (FIG. 5F). Together, these results suggest that the predicted stem-loop structure and the 5′ sequence of tRF^{31Asp2} are important for its biological activity.

### EXAMPLE 1.5

### Dependence of tRF Immunity on AGO2/3/7

Applicant tested whether tRF^{31Asp2} induces immune responses in the dcl or ago mutant backgrounds. All dcl and ago single mutants largely exhibited WT-level PR1 induction, but a triple mutant defective for AGO2 and its clade members (AGO3 and AGO7; ago2/3/7) showed significantly lower levels of PR1 induction (FIG. 5G). The ago2/3/7 pretreated with tRF^{31Asp2} also failed to display enhanced immunity (FIG. 5B). Thus, loading of tRF^{31Asp2} into AGO2 or its clade members may be necessary to trigger defense responses.

### EXAMPLE 1.6

### tRF-Induced Defense Genes

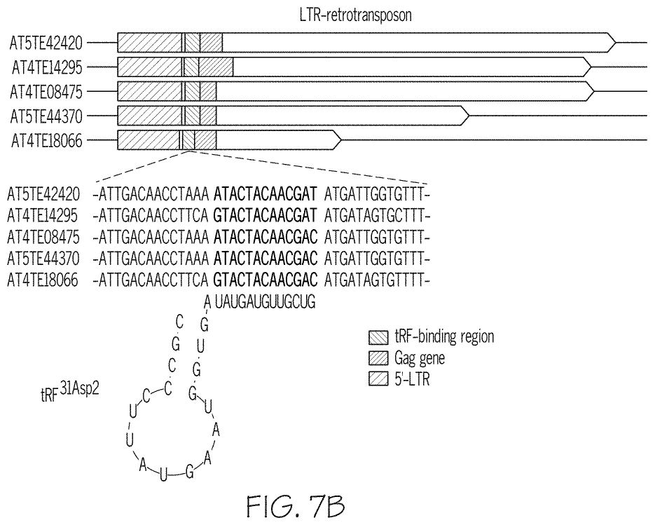

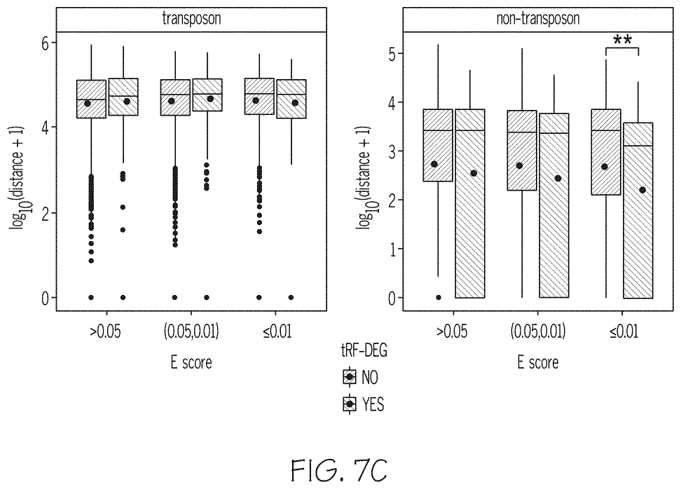

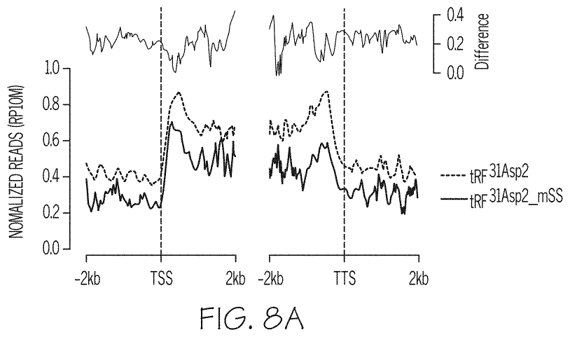

To identify tRF^{31Asp2}-induced DEGs, transcriptome analyses were performed. Comparing the transcriptomes of i) WT treated with tRF^{31Asp2} vs mock, ii) WT treated with tRF^{31Asp2} vs tRF^{31Asp2_mSS}, and iii) WT vs ago2/3/7 treated with tRF^{31Asp2} identified 810 DEGs present in all three combinations (tRF-DEGs, hereafter). The majority of these DEGs (503 of 810) also were identified as ETI-DEGs. Consistently, the transcriptome pattern for each of these comparisons and their average (FIG. 7A) showed predominant induction for ETI-DEGs (lower panels). In contrast, the remaining non-ETI-DEGs displayed a random pattern (upper panels).

### EXAMPLE 1.7

### tRF-Chromatin Association

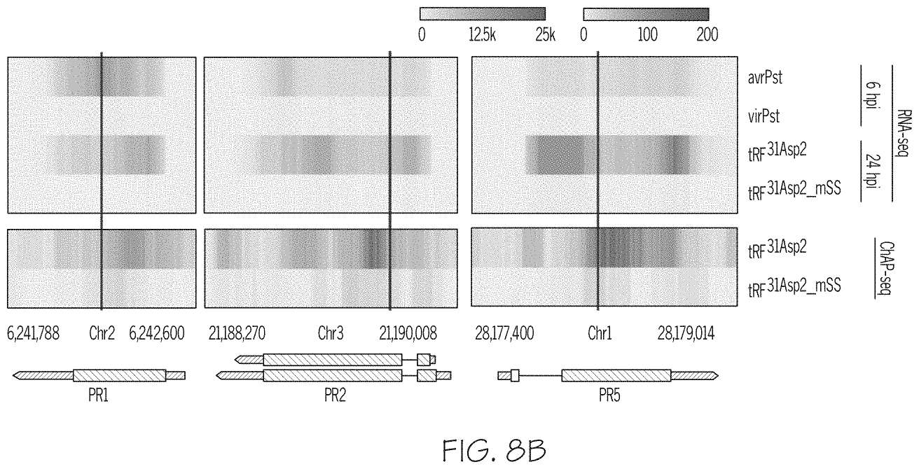

Applicant investigated the possibility that tRF^{31Asp2} physically interacts with Arabidopsis chromatin by infiltrating biotinylated tRF^{31Asp2} or tRF^{31Asp2_mSS} into WT leaves and performing ChAP (Chromatin Affinity Precipitation)-seq analysis. In WT, normalized reads showed that tRF^{31Asp2} interacted with chromatin significantly more than tRF^{31Asp2_mSS}, particularly around the genic area (FIG. 8A). Genomic regions exhibiting significantly greater interaction with tRF^{31Asp2} than tRF^{31Asp2_mSS} were identified using MACS2 program. The tRF^{31Asp2}-associated peaks were found in the genic area of many defense-associated genes, including PR1, PR2, and PR5 (FIG. 8B).

### EXAMPLE 1.8

### Gypsy's PBS and its Homologous Sequences, a Target of the tRF

Over 35% of ChAP-seq peaks localized to transposons. This represents a substantial enrichment, as transposons cover about 21% of the Arabidopsis genome. The greatest number of transposon-localized ChAP-seq peaks that also contained the tRF^{31Asp2} sequence, based on FASTA analysis with an E score of <0.01, were identified in the Gypsy superfamily of long-terminal repeat (LTR)-retrotransposons. Within this superfamily, ChAP-seq peaks with the tRF^{31Asp2} sequence were most prevalent in members of ATHILA2. The single-stranded 5′ region of tRF^{31Asp2} exhibited near-perfect complementarity to the Gypsy primer binding site (PBS) (FIG. 7B). Binding of Gypsy PBS by the 3′ end of tRNAs primes its proliferation. Since the 3′ end of Asp-2-tRNA^{GTC} is complementary to the 5′ sequences of tRF^{31Asp2}, tRF^{31Asp2}, might likewise bind these retrotransposons in a sequence-specific manner.

### EXAMPLE 1.9

### Proximity of tRF Sites to their DEGs

Applicant speculated that tRF^{31Asp2} might similarly regulate tRF-DEG expression via interaction with its complementary sequence, which might be located in recognizable or degenerated/unrecognizable transposons. To assess this possibility, the distance between genes and their nearest transposon-associated or -excluded ChAP-seq peak was assessed. For all genes, whether a tRF-DEG or not, the distance to the nearest transposon-associated peak was comparable (FIG. 7C, left panel). Without being bound by theory, the results are explainable because most annotated transposons are in gene-poor heterochromatin. However, analysis of ChAP-seq peaks lacking annotated transposons revealed that the peaks with a sequence homologous to tRF^{31Asp2} were significantly closer to tRF-DEGs than to non-tRF-DEGs with some overlapping with their gene bodies (FIG. 7C, right panel), suggesting that the tRF binding might be important in inducing neighboring tRF-DEGs.

### EXAMPLE 1.10

### Efficacy of Additional tRFs

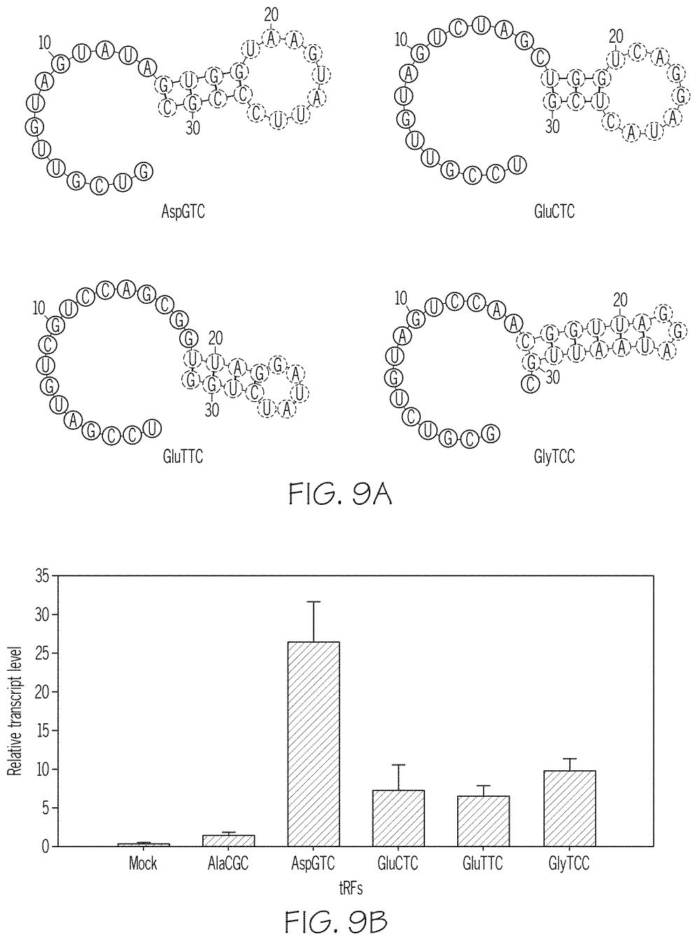

FIGS. 9A-9B illustrate that select 31 nt-long tRFs form secondary structures comparable to that of AspGTC and trigger the induction of a defense gene, PR1. FIG. 9A shows secondary structures of select tRF (31 nt long) sequences. FIG. 9B provides data related to the expression of PR1, as measured using real-time RT-PCR with PR1-specific primers relative to RHIP1, a housekeeping gene. Results are expressed as means±SE (n=5). AlaCGC used as a negative control in FIG. 9B did not display any secondary structure comparable to those in FIG. 9A.

### EXAMPLE 1.11

### Conclusions

In this Example, Applicant demonstrates that tRF^{31Asp2} is a DCL1-dependent sRNA rapidly induced well before the ETI transcriptomic peak. Treatment of tRF^{31Asp2} alone is sufficient to initiate immune responses robust enough to immunize Arabidopsis against virulent Pst and aphids. tRF^{31Asp2} also binds chromatin at a specific sequence present in Gypsy LTR-retrotransposons and defense genes. These observations suggest that this type of tRFs is likely one of the long-sought early signaling components in triggering and expediting transcriptional reprogramming in plant immunity via interacting with chromatin. Furthermore, the abundant presence of sRNAs derived from tRNAs and their likely interacting partners, LTR-retrotransposons, in all plant systems examined indicates that the positive regulatory role found herein may be ubiquitous. Additionally, the ability of tRF^{31Asp2} and its antisense nucleotides to enhance or suppress defense responses signifies that these molecules may be powerful tools for modulating stress responses.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of modulating a stress response in a eukaryotic organism against an environmental stress, said method comprising:
introducing an RNA fragment (RF) or an antisense nucleotide to said eukaryotic organism, wherein the RF comprises:
a sequence recognition site, wherein the sequence recognition site comprises a reverse complement sequence of a nucleotide sequence of the organism, and
a stem loop structure, wherein the stem loop structure comprises:
a paired region comprising paired RNA nucleotides, and
an unpaired region comprising unpaired RNA nucleotides in the form of a loop, and
wherein the antisense nucleotide comprises a nucleotide sequence that is complementary to the reverse complement sequence of the RF; and
wherein the RF or the antisense nucleotide modulates the stress response against the environmental stress.

2. The method of claim 1, wherein the introducing comprises introducing an RF to said eukaryotic organism.

3. The method of claim 2, wherein the RF is a transfer RNA (tRNA)-derived RNA fragment (tRF).

4. The method of claim 3, wherein the tRF lacks a tRNA anti-codon loop, a T-loop, an acceptor stem, and a variable loop, and wherein the sequence recognition site is in single-stranded form.

5. The method of claim 2, wherein the RF is selected from the group consisting of 5′ GUCGUUGUAGUAUAGUGGUAAGUAUUCCCGC-3′ (SEQ ID NO: 1), 5′-UCCGUUGUAGUCUAGCUGGUCAGGAUACUCG-3′ (SEQ ID NO: 2), UCCGAUGUCGUCCAGCGGUUAGGAUAUCUGG-3′ (SEQ ID NO: 3), 5′-GCGUCUGUAGUCCAACGGUUAGGAUAAUUGC-3′ (SEQ ID NO: 4), or a sequence with at least 75% identity with any one of SEQ ID NOS: 1-4.

6. The method of claim 2, wherein the RF comprises 5′ GUCGUUGUAGUAUAGUGGUAAGUAUUCCCGC-3′ (SEQ ID NO: 1) or a sequence with at least 75% identity to SEQ ID NO: 1, wherein 5′-GUCGUUGUAGUAUA-3′ represents the sequence recognition site, wherein 5′-GUGGUAAGUAUUCCCGC-3′ represents the stem loop structure, wherein 5′-GUGG-3′ pairs with 5′-CCGC-3′ to form the paired region of the stem loop structure, and wherein 5′-UAAGUAUUC-3′ represents the loop of the stem loop structure.

7. The method of claim 2, wherein the RF sequence recognition site comprises at least 10 bases, the RF paired region of the stem loop structure comprises at least 3 base pairs, and the RF unpaired region of the stem loop structure comprises at least 3 bases.

8. The method of claim 1, wherein the reverse complement sequence of the sequence recognition site binds to a retrotransposon region of the organism, wherein the retrotransposon region comprises a Gypsy and a Copia superfamily of long-terminal repeat (LTR)-retrotransposons.

9. The method of claim 1, wherein the eukaryotic organism is a plant or seed.

10. The method of claim 9, wherein the plant or seed is a dicot plant or seed selected from the group consisting of soybean, lettuce, tomato, potato, legumes, peas, beans, lentils, peanuts, cotton, Arabidopsis, and combinations thereof.

11. The method of claim 1, wherein the environmental stress is selected from the group consisting of abiotic stress, biotic stress, pathogenesis, pest infestation, and combinations thereof.

12. The method of claim 1, wherein the stress response comprises an immune response against the environmental stress.

13. The method of claim 1, wherein the environmental stress comprises pathogenesis, wherein the pathogenesis is caused by at least one pathogen, wherein the at least one pathogen comprises a bacterial pathogen, a fungal pathogen, a viral pathogen, or combinations thereof.

14. The method of claim 13, wherein the at least one pathogen comprises a bacterial pathogen selected from the group consisting of Erwinia, Pectobacterium, Pantoea, Agrobacterium, Pseudomonas, Ralstonia, Burkholderia, Acidovorax, Xanthomonas, Clavibacter, Streptomyces, Xylella, Spiroplasma, Phytoplasma, and combinations thereof.

15. The method of claim 1, wherein the RF or antisense nucleotide modulates the stress response by inducing the expression of one or more defense genes, wherein the defense genes comprise pathogenesis-related protein-1 (PR-1), Cyclic NUCLEOTIDE GATED CHANNEL 10 (CNGC10), GLUTAREDOXIN 13 (GRXS13), MAP KINASE 11 (MPK11), PLANT U-BOX 54 (PUB54), ACTIVATED DISEASE RESISTANCE 1 (ADR1), MYB DOMAIN PROTEIN 15 (MYB15), ACC synthase 6 (ACS6), Arabidopsis BAX INHIBITOR 1 (ATBI-1), Ca²⁺-DEPENDENT MODULATOR OF ICR1 (CMI1), CCR4-ASSOCIATED FACTOR 1A (AtCAF1a), DICARBOXYLATE CARRIER 2 (DIC2), ETHYLENE RESPONSE FACTOR 104 (ERF104), NDR1/HIN1-LIKE 3 (NHL3), NUDIX HYDROLASE HOMOLOG 21 (NUDT21), SALT TOLERANCE ZINC FINGER (STZ), THIOREDOXIN H-TYPE 5 (ATHS), CALMODULIN-LIKE 24 (CML24), analogs thereof, homologs thereof, and combinations thereof.

16. The method of claim 1, wherein the method is utilized to treat or prevent the effect of the environmental stress in the eukaryotic organism.

17. The method of claim 1, wherein the RF or antisense nucleotide modulates the stress response by increasing the stress response against the environmental stress.

18. The method of claim 1, wherein the RF or antisense nucleotide modulates the stress response by decreasing the stress response against the environmental stress.

19. The method of claim 1, wherein the introducing comprises introducing an antisense nucleotide to said eukaryotic organism.

20. The method of claim 19, wherein the antisense nucleotide is an RNA or DNA nucleotide selected from the group consisting of a reverse complement sequence of SEQ ID NO: 1, 5′-GCGGGAAUACUUACCACUAUACUACAACGAC-3′ (SEQ ID NO: 5), 5′-GCGGGAATACTTACCACTATACTACAACGAC-3′ (SEQ ID NO: 6), an antisense nucleotide for a sequence recognition site of SEQ ID NO: 1, 5′-GTCGTTGTAGTAT-3′ (SEQ ID NO: 7), 5′-GUCGUUGUAGUAU-3′ (SEQ ID NO: 8), a reverse complement sequence of SEQ ID NO: 2, 5′-CGAGUAUCCUGACCAGCUAGACUACAACGGA-3′ (SEQ ID NO: 9), 5′-CGAGTATCCTGACCAGCTAGACTACAACGGA-3′ (SEQ ID NO: 10), an antisense nucleotide for a sequence recognition site of SEQ ID NO: 2, 5′-GCUAGACUACAACGGA-3′ (SEQ ID NO: 11), 5′-GCTAGACTACAACGGA-3′ (SEQ ID NO: 12), a reverse complement sequence of SEQ ID NO: 3, 5′-CCAGAUAUCCUAACCGCUGGACGACAUCGGA-3′ (SEQ ID NO: 13), 5′-CCAGATATCCTAACCGCTGGACGACATCGGA-3′ (SEQ ID NO: 14), an antisense nucleotide for a sequence recognition site of SEQ ID NO: 3, 5′-CCGCUGGACGACAUCGGA-3′ (SEQ ID NO: 15), 5′-CCGCTGGACGACATCGGA-3′ (SEQ ID NO: 16), a reverse complement sequence of SEQ ID NO: 4, 5′-GCAAUUAUCCUAACCGUUGGACUACAGACGC-3′ (SEQ ID NO: 17), 5′-GCAATTATCCTAACCGTTGGACTACAGACGC-3′ (SEQ ID NO: 18), an antisense nucleotide for a sequence recognition site of SEQ ID NO: 4, 5′-UUGGACUACAGACGC-3′ (SEQ ID NO: 19), and 5′-TTGGACTACAGACGC-3′ (SEQ ID NO: 20).

21. An isolated RNA fragment (RF) comprising:
a sequence recognition site, wherein the sequence recognition site comprises a reverse complement sequence of a nucleotide sequence of an organism, and
a stem loop structure, wherein the stem loop structure comprises:
a paired region comprising paired RNA nucleotides, and
an unpaired region comprising unpaired RNA nucleotides in the form of a loop; and
wherein the RF modulates the stress response against an environmental stress.

22. The RF of claim 21, wherein the RF is a transfer RNA (tRNA)-derived RNA fragment (tRF).

23. The RF of claim 22, wherein the tRF lacks a tRNA anti-codon loop, a T-loop, an acceptor stem, and a variable loop, and wherein the sequence recognition site is in single-stranded form.

24. The RF of claim 21, wherein the RF is selected from the group consisting of 5′ GUCGUUGUAGUAUAGUGGUAAGUAUUCCCGC-3′ (SEQ ID NO: 1), 5′-UCCGUUGUAGUCUAGCUGGUCAGGAUACUCG-3′ (SEQ ID NO: 2), UCCGAUGUCGUCCAGCGGUUAGGAUAUCUGG-3′ (SEQ ID NO: 3), 5′-GCGUCUGUAGUCCAACGGUUAGGAUAAUUGC-3′ (SEQ ID NO: 4), or a sequence with at least 75% identity with any one of SEQ ID NOS: 1-4.

25. The RF of claim 21, wherein the RF comprises 5′ GUCGUUGUAGUAUAGUGGUAAGUAUUCCCGC-3′ (SEQ ID NO: 1) or a sequence with at least 75% identity to SEQ ID NO: 1, wherein 5′-GUCGUUGUAGUAUA-3′ represents the sequence recognition site, wherein 5′-GUGGUAAGUAUUCCCGC-3′ represents the stem loop structure, wherein 5′-GUGG-3′ pairs with 5′-CCGC-3′ to form the paired region of the stem loop structure, and wherein 5′-UAAGUAUUC-3′ represents the loop of the stem loop structure.

26. The RF of claim 21, wherein the RF sequence recognition site comprises at least 10 bases, the RF paired region of the stem loop structure comprises at least 3 base pairs, and the RF unpaired region of the stem loop structure comprises at least 3 bases.

27. The RF of claim 21, wherein the reverse complement sequence of the sequence recognition site binds to a retrotransposon region of the organism, wherein the retrotransposon region comprises a Gypsy and a Copia superfamily of long-terminal repeat (LTR)-retrotransposons.

28. An isolated antisense nucleotide, wherein the antisense nucleotide is an RNA or DNA nucleotide selected from the group consisting of a reverse complement sequence of SEQ ID NO: 1, 5′-GCGGGAAUACUUACCACUAUACUACAACGAC-3′ (SEQ ID NO: 5), 5′-GCGGGAATACTTACCACTATACTACAACGAC-3′ (SEQ ID NO: 6), an antisense nucleotide for a sequence recognition site of SEQ ID NO: 1, 5′-GTCGTTGTAGTAT-3′ (SEQ ID NO: 7), 5′-GUCGUUGUAGUAU-3′ (SEQ ID NO: 8), a reverse complement sequence of SEQ ID NO: 2, 5′-CGAGUAUCCUGACCAGCUAGACUACAACGGA-3′ (SEQ ID NO: 9), 5′-CGAGTATCCTGACCAGCTAGACTACAACGGA-3′ (SEQ ID NO: 10), an antisense nucleotide for a sequence recognition site of SEQ ID NO: 2, 5′-GCUAGACUACAACGGA-3′ (SEQ ID NO: 11), 5′-GCTAGACTACAACGGA-3′ (SEQ ID NO: 12), a reverse complement sequence of SEQ ID NO: 3, 5′-CCAGAUAUCCUAACCGCUGGACGACAUCGGA-3′ (SEQ ID NO: 13), 5′-CCAGATATCCTAACCGCTGGACGACATCGGA-3′ (SEQ ID NO: 14), an antisense nucleotide for a sequence recognition site of SEQ ID NO 3, 5′-CCGCUGGACGACAUCGGA-3′ (SEQ ID NO: 15), 5′-CCGCTGGACGACATCGGA-3′ (SEQ ID NO: 16), a reverse complement sequence of SEQ ID NO: 4, 5′-GCAAUUAUCCUAACCGUUGGACUACAGACGC-3′ (SEQ ID NO: 17), 5′-GCAATTATCCTAACCGTTGGACTACAGACGC-3′ (SEQ ID NO: 18), an antisense nucleotide for a sequence recognition site of SEQ ID NO: 4, 5′-UUGGACUACAGACGC-3′ (SEQ ID NO: 19), 5′-TTGGACTACAGACGC-3′ (SEQ ID NO: 20), and a sequence with at least 75% identity with any one of SEQ ID NOS: 5-20.
